(19) United States
(12) Patent Application Publication
(10) Pub. No.: US 2013/0220333 A1
(43) Pub. Date: Aug. 29, 2013

(54) TRACHEAL TUBE WITH CUSTOMIZABLE FLANGES

(71) Applicant: Sean Morris, Ballymurray (IE)

(72) Inventor: Sean Morris, Ballymurray (IE)

(73) Assignee: Nellcor Puritan Bennett LLC, Boulder, CO (US)

(21) Appl. No.: 13/405,008

(22) Filed: Feb. 24, 2012

(51) Int. Cl. A61M 16/04

(52) U.S. Cl. 128/207.17

(57) ABSTRACT

A tracheal tube assembly includes a cannula configured to be positioned in a patient airway and a customizable flange configured to be secured about the cannula. The customizable flange further includes a first flange portion and a second flange portion. The second flange portion is configured to be selectively removed from the customizable flange to customize the length of the customizable flange.

### BACKGROUND

The present disclosure relates generally to the field of tracheal tubes and, more particularly, to a tracheal tube flange having customizable flanges.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening.

The opening created by the tracheal tube may be utilized to define a clear passageway for air, other gases, and medicaments to the trachea and lungs, thus providing an artificial airway for spontaneous or mechanical ventilation of a patient. Such tracheal tubes may include endotracheal tubes and tracheostomy tubes. When the inner or distal end of the tracheostomy tube is properly inserted into the trachea, the tracheostomy tube may be secured in place by flanges (e.g., “wings”). The flanges may be positioned on the neck and may then be secured in placed by a strap such as a ribbon or soft tie. However, in patients having certain neck obstructions and/or having unusual anatomies, the flanges may not be suitable for their intended purpose. There is a need, therefore, for improved tracheal tubes, and particularly for improved tracheostomy tube flanges. It would be desirable to provide a tube flange that is customizable for a variety of anatomies.

### BRIEF DESCRIPTION

This disclosure provides a novel tracheal tube having customizable flanges designed to respond to such needs. The tube flanges allow for a tailored fit at the clinical site, suitable for conforming to a variety of patient sizes and anatomies. Indeed, instead of submitting a special modification to the manufacturer, a clinician or user may customize the flange to fit a particular patient anatomy. In a tracheostomy tube embodiment, for example, the customizable flange fits adjacent to the neck of a patient and certain customizable features are provided, including areas with perforations and/or areas of reduced mechanical strength. The clinician or user may cut the flange to a desired shape and/or size by using the aforementioned areas. For example, the flange may be cut to a shape having a geometry suitable for avoiding a neck protrusion, or at a size suitable for comfortably fitting to a variety of patients.

In another embodiment, the customizable flange may include a “clip-on” flange member, such as a manually removable and replaceable member of the flange, as described in more detail below. Various flange members of different sizes and shapes may be provided, suitable for coupling with a receiving member of the customizable flange. Accordingly, the clinician or user may select a flange member more suitable for a given patient anatomy. The selected flange member may be manually secured to the flange, thus customizing the flange to comfortably fit the patient. Flange members of various sizes and shapes may be provided as a kit, thus enabling a custom fit at the clinical site. Indeed, the customizable flanges may allow for a proper positioning of the tracheal tube in a wide range of patients, including pediatric and adult patients. Different colors of flanges, flange portions, and cannulae may also be provided, useful in identifying parts as well as in improving aesthetics and providing for different styling options.

The systems disclosed herein also include a removable flange with flange features allowing the flange to be placed onto the patient's neck, prior to insertion, giving the clinician or user an opportunity to draw a profile onto the flange, and/or to assess a shape on the flange that may make the flange more comfortable while in use after insertion.

Thus, in accordance with a first aspect, a tracheal tube assembly includes a cannula configured to be positioned in a patient airway and a customizable flange configured to be secured about the cannula. The customizable flange further includes a first flange portion and a second flange portion. The second flange portion is configured to be selectively removed from the customizable flange to customize the length of the customizable flange.

In accordance with another aspect, a tracheal tube assembly includes a cannula configured to be positioned in a patient airway and a customizable flange configured to be secured about the cannula. The customizable flange further includes a receiving member and a first flange member. The first flange member is configured to be coupled to the receiving member to customize the length, the width, or a combination thereof, of the customizable flange.

Also disclosed herein is a tracheal tube assembly including a cannula configured to be positioned in a patient airway and a customizable flange configured to be secured about the cannula. The customizable flange comprises a first length and a first width. The customizable flange customizable flange is configured to be customized to a second length, a second width, or a combination thereof.

### BRIEF DESCRIPTION OF THE DRAWINGS

Various aspects of the disclosed techniques may become apparent upon reading the following detailed description and upon reference to the drawings in which:

FIG. 1 is a perspective view of an exemplary tracheal tube assembly in accordance with aspects of the present techniques;

FIG. 2 is a view of the tracheal tube assembly of FIG. 1 having a plurality of tracheal flange indentations;

FIG. 3 is a view of the tracheal tube assembly of FIG. 2 with a tracheal flange portion removed;

FIG. 4 is a view of the tracheal tube assembly of FIG. 1 having a plurality of tracheal flange openings or through holes;

FIG. 5 is a view of the tracheal tube assembly of FIG. 4 with tracheal flange portions removed;

FIG. 6 is a view of the of the tracheal tube assembly of FIG. 1, including tracheal flange portions having different mechanical strengths;

FIG. 7 is a view of the tracheal tube assembly of FIG. 6 with a tracheal flange portion removed;

FIG. 8 is a view of the of the tracheal tube assembly of FIG. 1, including replaceable tracheal flange members and receiving members;

FIG. 9 is a first view of a replaceable tracheal flange member and a receiving member of FIG. 8;

FIG. 10 is a second view of a replaceable tracheal flange member and a receiving member of FIG. 8;

FIG. 11 is a third view of a replaceable tracheal flange member and a receiving member of FIG. 8;

FIG. 12 is a view of a replaceable tracheal flange member and a receiving member, and;

FIG. 13 is a view of a replaceable tracheal flange member and a receiving member.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

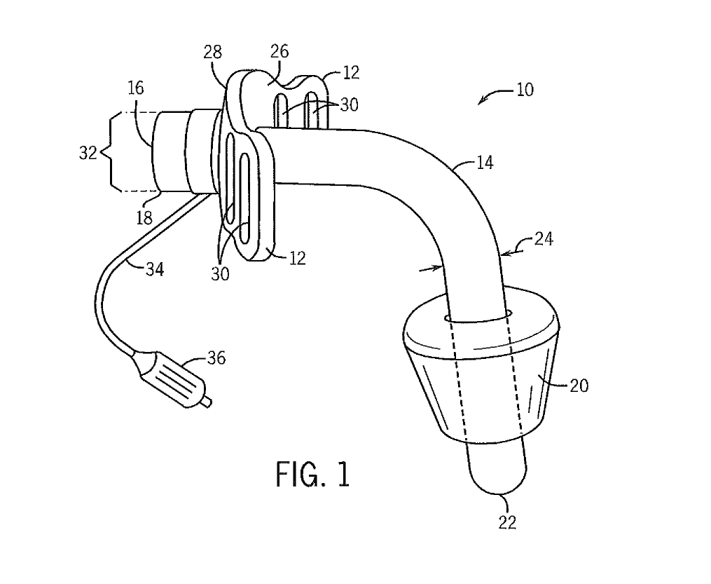

A tracheal tube according to a preferred embodiment is illustrated in FIG. 1. The tracheal tube assembly 10 represented in the figures is a tracheostomy tube, although aspects of this disclosure could be applied to other tracheal tube structures, such as endotracheal tubes. The application to a tracheostomy tube is apt, however, insomuch as such tubes tend to be worn for longer periods of time, and thus may include customizable flanges 12 disposed on a patient's neck and useful in comfortably maintaining a secure airway connection.

A cannula 14 is illustrated extending both distally as well as proximally from the flange 12. The cannula 14 may include an opening 16 disposed on a proximal end of an end connector 18. During intubation, the tracheal tube assembly 10 is placed through an opening formed in the neck and trachea of a patient, and extending into the patient airway. The embodiment illustrated in the figures includes a sealing cuff 20, although in practice a wide range of tube designs may be used, including tubes having no cuffs or tubes having multiple cuffs around the outer cannula 14. The cannula 14 in the illustrated embodiment forms a conduit from which liquids or gases, including medications, may enter through the proximal opening 16 an exit through a distal opening 22. The cannula 14 has an outer dimension 24 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, outer dimension 24 of the cannula 14 may range from 4 mm to 16 mm.

In one embodiment, the outer cannula 14 enters the flange 12 along a lower face 26 and protrudes through an upper face 28 of the flange 12. When in use, the face 26 will generally be positioned against the neck of a patient, with the cannula extending through an opening formed in the neck and trachea. The flanges 12 extend laterally and serve to allow a strap or retaining member (not shown) to hold the tube assembly in place on the patient. In the illustrated embodiment, apertures 30 are formed in each side flange 12 to allow the passage of such a retaining device. In many applications, the flange 12 may be taped or sutured in place as well.

The end connector 18 is formed in accordance with industry standards to permit and facilitate connection to ventilating equipment (not shown). By way of example, standard outer dimensions may be provided as indicated at reference numeral 32 that allow a mating connector piece to be secured on the connector shown. By way of example, a presently contemplated standard dimension 32 accommodates a 15 mm connector, although other sizes and connector styles may be used. In use, then, air or other gas may be supplied through the connector and the cannula 14, and gases may be extracted from the patient. For example, the tube assembly 10 may be inserted into the patient's airways, and the cuff 20 may then be inflated through an inflation lumen 34. A pilot balloon 36 may then indicate that air is in the cuff 20, thus sealing the patient's airway. Once the tracheal tube is positioned and secured, a ventilator may be coupled to the end connector 18 to provide for respiratory support.

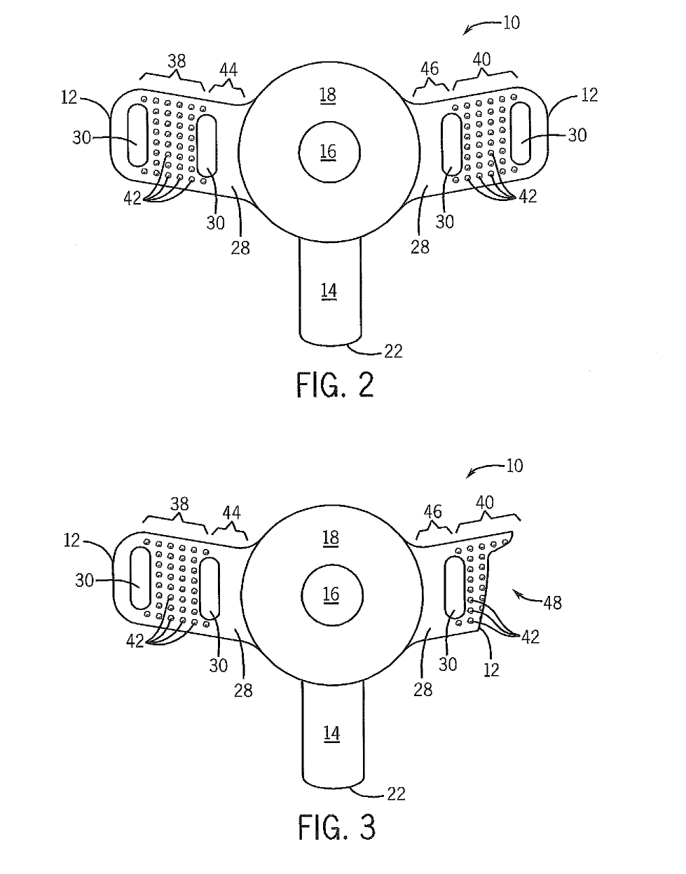

Certain portions of the flange 12, as described in more detail below with respect to FIG. 2, enable the customization of the flange 12. More specifically, in the example tracheal tube assembly 10 depicted in FIG. 2, the flanges 12 are illustrated as including customizable portions 38 and 40 having lessened mechanical strength. In the depicted example, the portions 38 and 40 both include a plurality of indentations or dimples 42. That is, the portions 38 and 40 have less material at each indentation 42 when compared to, for example, portions 44 and 46 of the flange 12. In one manufacturing embodiment, the indentations 42 may be molded or overmolded onto the flanges 12. In another manufacturing embodiment, the indentations 42 may be milled (e.g., machine milled, CNC milled) onto the flanges 12. The amount of material present in the indentations 42 may be between 70%-90%, 50%-80%, 1%-99% of the material present in the portions 44 and 46 of the flanges 12. The flange 12, including the portions 38, 40, 44, and 46, may be manufactured from acrylonitrile butadiene styrene (ABS), polyvinyl chloride (PVC), phthalate-free PVC, polyethylene terephthalate (PET), low-density polyethylene (LDPE), polypropylene, silicone, neoprene, and/or polyisoprene.

The indentations 42 may be manufactured in an approximately circular shape, as depicted, or in other geometric shapes (e.g., squares, rectangles, triangles). Non-geometric shapes may also be used. By providing for the indentations 42, the portions 38 and 40 may be of lesser mechanical strength when compared to other portions (e.g., 44, 46) of the flanges 12. Accordingly, the clinician or user may comfortably fit the flanges 12 to a desired shape, for example, by cutting a desired shape at the portions 38 and/or 40, as described in more detail below with respect to FIG. 3.

FIG. 3 illustrates an embodiment of the tracheal tube assembly 10 having a section 48 of the portion 40 excised or otherwise removed, for example, to improve the fit of the flanges 12 when disposed against a patient's neck anatomy. As mentioned above, the section 48 may be removed from portion 40 with the aid of the indentations 42. By providing for less material strength, the indentations 42 may substantially reduce the level of effort used in removing the portion 48. For example, a scalpel, trauma shears (e.g. paramedic shears), scissors, and the like, may be used to cut the section 48 from the portion 40. By cutting through the indentations 42, the flanges 12 may be customized in a plurality of shapes (e.g., geometric, non-geometric) and/or sized suitable to fit a variety patient anatomies.

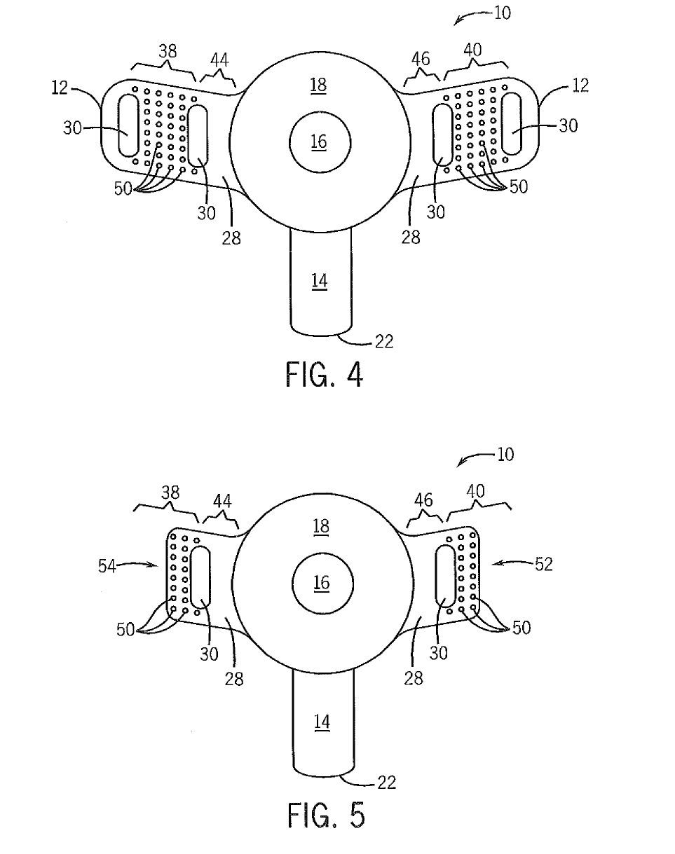

In the depicted embodiment, the portion 48 may have been removed, for example, for resizing purposes and/or to overcome obstructions in the neck area, such as a catheter (e.g., central venous catheter), sutures, a medical instrument (e.g., endoscope), anatomical protrusions, and so forth. While portion 38 is depicted as unexcised, a clinician or user may, of course, also remove a section of the portion 38. The clinician or user may then insert a strap or retaining member through the openings or apertures 30 to secure the tracheal tube assembly 10 to the patient's neck. In other embodiments, the lessened mechanical strength at portions 38 and or 40 may be provided by through holes or openings, as depicted in FIG. 4.

In the illustrated embodiment depicted in FIG. 4, the portions 38 and 40 include through holes or openings 50 rather than the indentations 42 of FIGS. 2 and 3. In other embodiments, the indentations 42 may be combined with the through holes 50. Because the through holes 50 include less material in the portions 44 and 46 when compared to the indentations 42, the use of the through holes 50 may provide for less effort when excising or otherwise removing sections from the portions 38 and/or 40. The through holes 50 may be molded, overmolded, milled, and/or drilled into the portions 38 and 40. It is to be noted that, while FIG. 4 depicts a total of 4 apertures 30 with a portion 38 or 40 disposed between the apertures 30, other embodiments may include 6, 8, 10 or more apertures 30, with portions between the apertures 30 having indentations 42, through holes 50, or a combination thereof.

As mentioned above, the tracheal tube assembly 10 may be customized to fit a variety of anatomies and sizes, for example, by cutting at the portions 38 and 40 to reshape or customize the tracheal flange 12. FIG. 5 depicts the tracheal tube assembly 10 embodiment shown in FIG. 4 where sections 52 and 54 have been removed to more comfortably fit a smaller neck anatomy. The clinician may, for example, measure the patient's neck diameter, and then use the measurement to derive a desired size and shape for the flange 12. The clinician may then use a cutting instrument (e.g., scalpel, EMT shear, scissors) to excise the portions 50 and 52. By providing for reduced mechanical strength compared to other portions of the tracheal flange 12, the portions 38 and 40 may enable a more efficient removal of the portions 52 and 54. It is to be noted that the positioning or “seating” of the tracheal tube assembly 10 can be substantially improved by all of the embodiments disclosed herein. Indeed, by customizing the portions 38 and 40, and/or by using the various removable flange embodiments, the tracheal tube assembly 10 may be customized to be positioned more comfortably on a patient, alternatively to or additionally to changing flange 12 sizes.

The cannula 14 may then be inserted into the trachea, and the flanges 12 may be positioned on the neck. A retaining member (not shown), such as a soft tie, may then be inserted through the apertures 30 and used to securely fasten the tracheal tube assembly 10 to the patient. In this manner, a more custom fit of the tracheal tube assembly 10 for a specific patient anatomy may be provided.

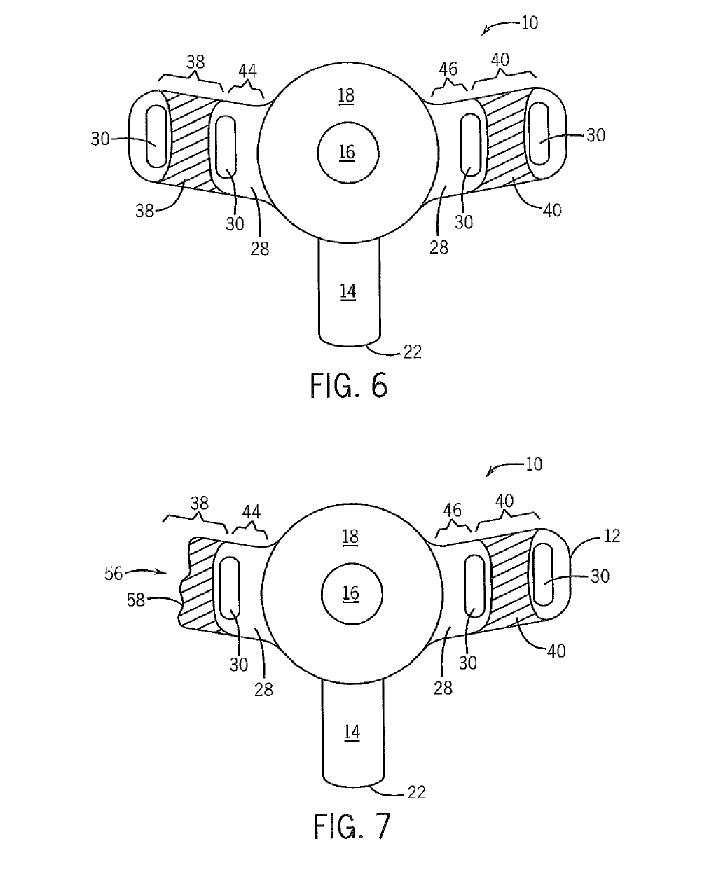

In an embodiment depicted in FIG. 6, the portions 38 and 40 may not include the indentations 42 or the through holes 50, but may be manufactured out of a material having a lower mechanical strength than, for example, the remainder sections of the flanges 12. For example, the portions 38 and 40 may be manufactured out of a soft silicone, a neoprene, or may be manufactured to include less material than the remainder sections of the flanges 12. For example, the portions 38 and 40 may be made thinner when compared to the remainder sections of the flanges 12. In certain embodiments, the portions 38 and 40 may also be colored differently than other components of the tracheal tube assembly 10. For example, the portions 38 and 40 may be provided in a first color (e.g., red, orange, yellow, green, blue, indigo, violet) while the remainder portions of the flanges 12 may be provided in a second color (or left uncolored). In other embodiments, the portions 38 and 40 may include markings, such as lines, dots, dashes, and so on, denoting the boundaries of the portions 38 and 40. Accordingly, the clinician or user may more easily visualize the portions 38 and 40 for subsequent customization of the flanges 12.

FIG. 7 illustrates the tracheal tube 10 embodiment of FIG. 6 where a section 56 of the flange 12 has been removed. More specifically, the figure illustrates a cut having a shape 58 through the portion 38 of the flange 12. The shape 58 may include curves, lines, geometric figures (e.g., circles, squares), non-geometric figures, and/or or other topological constructs. A clinician may, for example, first draw the shape 58 onto the portion 38 and then cut the shape 58 by using, a scalpel, a shear, or scissors. Any type of shape may be cut at the portion 38 or 40, suitable for customizing the flange 12 for a specific anatomy, or to avoid obstructions.

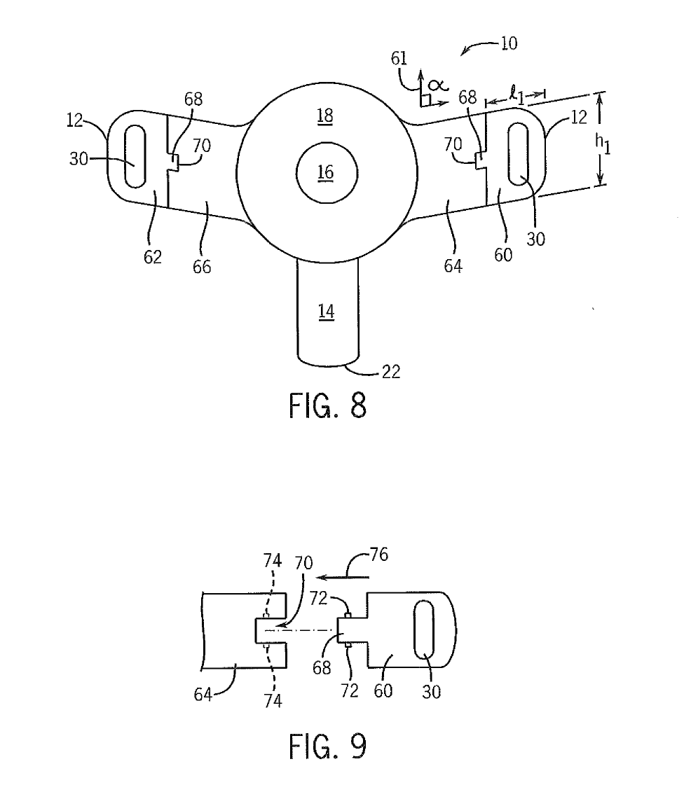

Turning now to FIG. 8, the figure depicts an embodiment of the tracheal tube assembly 10 providing customizable flanges 12 by including replaceable flange members 60 and 62. In the depicted embodiment, the flange members 60 and 62 may be manually removed and replaced with other flange members having different dimensions, including a different length l, width w, and/or height h. Other flange members may also incorporate a different shape (e.g., different angle α). For example, while the depicted embodiment shows the flange member 60 projecting outwardly from a cannula's axis 61 at an acute angle α, other embodiments may provide for a straight angle α, or an obtuse angle α. The various flanges members may be provided as a kit. By including a variety of different flange members having different lengths, widths, heights, and angles α, the clinician or user may select members that more comfortably fit a given patient anatomy. For example, the clinician may measure the neck diameter of the patient, visually identify other features on the neck area (e.g., clinical attachments), and then select flange members having a desired size and/or angle α. The clinician may then manually assemble the tracheal tube assembly 10 in situ by attaching the selected flange members 60 and 62 into receiving members 64 and 66 of the tracheal tube assembly 10.

In the illustrated embodiment, the flange members 60 and 62 include a rectangular protrusion 68 useful in securing the flange members 60 and 62 to the receiving members 64 and 66. The receiving members 64 and 66 include a receptacle 70 having a shape designed to receive or “mate” with the protrusion 68, as described in more detail below with respect to FIG. 9. Accordingly, the clinician or user may more easily select and manually insert the flange member 60 and 62 into the receiving members 64 and 66.

FIG. 9 depicts an embodiment of the flange member 60 shown in FIG. 8 prior to being coupled to the receiving member 64. As mentioned above, the protrusion 68 may be disposed inside the receptacle 70 of the receptacle member 64. By providing for the receptacle 70 shaped to mate with the projection 68, the receiving member 64 enables a secure manual fastening using less effort and time. In the depicted embodiment, the protrusion 68 includes an approximately rectangular shape, while the receptacle 70 includes a complementary approximately rectangular shape suitable for guiding and enveloping the protrusion 68. In certain embodiments, the protrusion 68 may include spring-biased projections or “pegs” 72, suitable for engaging holes 74 included in the receiving member 64. For example, the clinician may manually insert the protrusion 68 inwardly into the receptacle 70 in a direction 76 until the projections 72 are disposed inside of the holes 74.

In some embodiments, an audible noise, such as a click, may denote the secure engagement of the projections 72 inside the holes 74. In another embodiment, the projections 72 and holes 74 may not be used. In this embodiment, and interference fit or press fit created by coupling the flange member 60 to the receiving member 64 securely fastens the components 60 and 64 to each other. However, the interference fit may enable a clinician or user to apply a manual force suitable for separating the components 60 and 64. Accordingly, the clinician or user may manually attach or remove the flange member 60 from the receiving member 64, when desired.

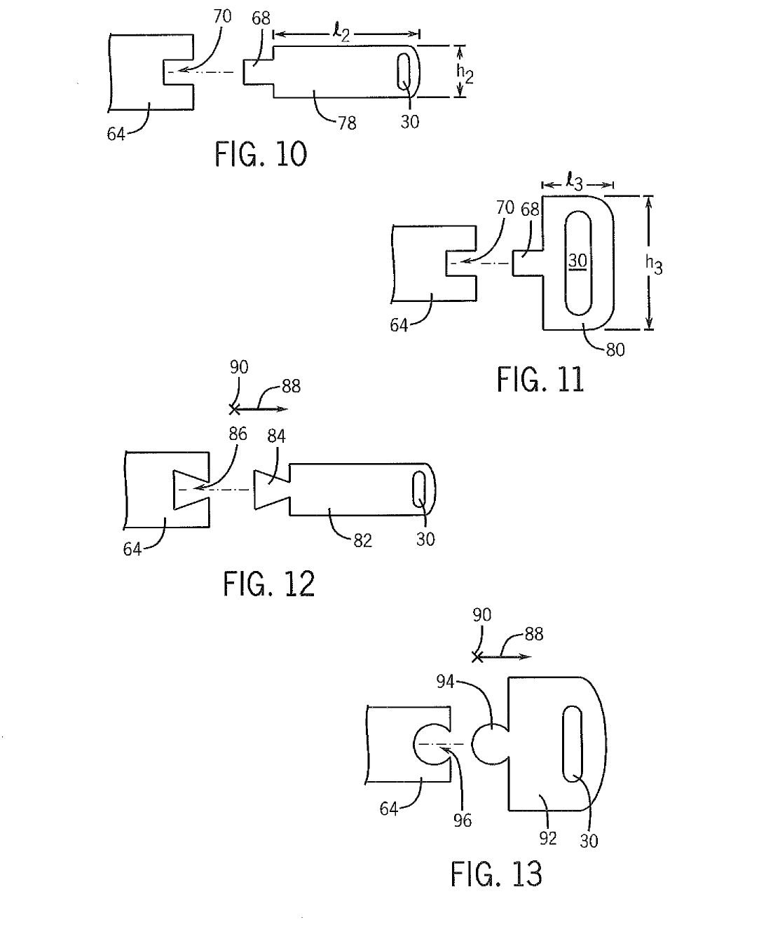

In other embodiments, such as the embodiments show in FIGS. 10 and 11, the flange members may be provided in different sizes and shapes. In the depicted example of FIG. 10, a flange member 78 is illustrated, having a length l2 sized larger than the length l1, and a height h2 smaller than the height h1 of the flange member 60. In the embodiment of FIG. 11, a flange member 80 is illustrated, having a length 13 sized smaller than the length l1, and a height h3 smaller than the height h1 of the flange member 60. By including a variety of sizes and shapes, for example, as a kit, the tracheal tube assembly 10 may be assembled at the clinical site to comfortably fit a variety of neck anatomies and shapes.

In other embodiments, such as the embodiments show in FIGS. 12 and 13, other shapes (e.g., geometric shapes, non-geometric shapes) may be provided, suitable for securing flange members to the tracheal tube assembly 10. In the depicted example of FIG. 12, a flange member 82 is depicted as having a triangular-shaped protrusion 84. The triangular protrusion 84 may be inserted into a triangular receptacle 86 of the receiving member 64. The triangular shapes of the protrusion 84 and the receptacle 86 may provide for a more secure attachment of the flange member 82 to the receiving member 64. These triangular shapes may be particularly useful in resisting a pulling force 88 from separating the flange member 82 from the receiving member 64. For example, the triangle shapes may provide added resistive force by “wedging” the protrusion 84 to the receptacle 86 when the pulling force 88 is exerted.

During in situ assembly, the clinician or user may place the flange member 82 on top of the receiving member 64 so that the protrusion 84 is directly over the receptacle 86. The flange member 82 and receiving member 64 may then be manually pressed inwardly towards each other, for example, by using a thumb and an index finger, thus inserting the protrusion 84 into the receptacle 86. An interference fit between the flange member 82 and receiving member 64 may securely fasten the components 82 and 64 to each other. Additionally, projections, such as the projections 72 depicted in FIG. 9, may be included in the protrusion 84, with corresponding openings 74 included in the receptacle 86. During removal, a manual force, such as a force 90 depicted as a normal vector to the force 88 may be applied, thus disengaging the flange member 82 from the receiving member 64. In this manner, the flange member 82 may be easily attached and/or replaced from the receiving member 64.

FIG. 13 depicts the use of circular shapes, in addition to rectangular and/or triangular shapes, suitable for securing flange members to the tracheal tube assembly 10. In the depicted example of FIG. 13, a flange member 92 is depicted as having a circular-shaped protrusion 94. Similar in use to the rectangular and triangular protrusions 68 and 84, the circular-shaped protrusion 94 may be inserted into a circular receptacle 96 of the receiving member 64. The circular shapes of the protrusion 94 and the receptacle 96 may also provide for added security of the attachment of the flange member 92 to the receiving member 64. These circular shapes may also be particularly useful in resisting a pulling force 88 from separating the flange member 92 from the receiving member 64.

The clinician or user may also manually attach or remove the flange member 92. For example, during in situ assembly, the clinician or user may place the flange member 92 on top of the receiving member 64 so that the protrusion 94 is directly over the receptacle 96. The flange member 92 and receiving member 64 may then be manually pressed inwardly towards each other, thus inserting the protrusion 94 into the receptacle 96. An interference fit between the flange member 92 and receiving member 64 may securely fasten the components 92 and 64 to each other. During removal, a manual force, such as the force 90 depicted as a normal vector to the force 88 may be applied, thus disengaging the flange member 92 from the receiving member 64. In this manner, the flange member 92 may be easily attached and/or removed from the receiving member 64.

What is claimed is:

1. A tracheal tube assembly comprising:
a cannula configured to be positioned in a patient airway;
a customizable flange configured to be secured about the cannula, the customizable flange comprising:
a first flange portion; and
a second flange portion configured to be selectively removed from the customizable flange to customize the length of the customizable flange.

2. The assembly of claim 1, wherein the first flange portion comprises a first mechanical strength greater than a second mechanical strength included in the second flange portion.

3. The assembly of claim 1, wherein the second flange portion comprises a plurality of indentations.

4. The assembly of claim 1, wherein second flange portion comprises at least 10% less material than the first flange portion.

5. The assembly of claim 1, wherein second flange portion comprises a plurality of through holes.

6. The assembly of claim 1, wherein the first flange portion comprises a first material having greater mechanical strength than a second material included in the second flange portion, and wherein the first material is different from the second material.

7. The assembly of claim 6, wherein the first material comprises an acrylonitrile butadiene styrene (ABS), a polyvinyl chloride (PVC), a phthalate-free PVC, a polyethylene terephthalate (PET), a low-density polyethylene (LDPE), polypropylene, a silicone, a neoprene, a polyisoprene, or a combination thereof, and wherein the first flange portion comprises a first color and the second flange portion comprises a second color.

8. The assembly of claim 1, wherein the second flange portion is configured to be cut in a shape having a curve, a line, or a combination thereof.

9. The assembly of claim 1, wherein the first portion comprises a first opening, and wherein the second portion comprises a second opening, and wherein the first opening and the second opening are configured to insert a retaining member to secure the tracheal tube assembly to a neck.

10. A tracheal tube assembly comprising:
a cannula configured to be positioned in a patient airway;
a customizable flange configured to be secured about the cannula, the customizable flange comprising:
a receiving member; and
a first flange member configured to be coupled to the receiving member to customize a first length, a first width, or a combination thereof, of the customizable flange.

11. The assembly of claim 10, comprising a second flange member having a second length, a second width, or a combination thereof, and wherein the second flange member is configured to be coupled to the receiving member to replace the first flange member.

12. The assembly of claim 11, wherein the cannula, the receiving member, the first flange member, and the second flange member are included in a kit.

13. The assembly of claim 10, wherein the receiving member comprises a receptacle and wherein the first flange member comprises a protrusion configured to be manually inserted into the receptacle.

14. The assembly of claim 13, wherein the receptacle comprises an opening, and wherein the protrusion comprises a spring-biased projection configured to be inserted into the opening.

15. The assembly of claim 13, wherein the protrusion comprises a geometric-shaped protrusion and wherein the receptacle comprises a geometric-shaped receptacle.

16. The assembly of claim 15, wherein the geometric shaped protrusion comprises a rectangle, a triangle, a circle, or a combination thereof.

17. A tracheal tube assembly comprising:
a cannula configured to be positioned in a patient airway; and
a customizable flange configured to be secured about the cannula, wherein the customizable flange comprises a first length and a first width, and wherein the customizable flange is configured to be customized to a second length, a second width, or a combination thereof.

18. The assembly of claim 17, wherein the customizable flange comprises a flange portion configured to be removed from the customizable flange to customize the second length, the second width, or the combination thereof.

19. The assembly of claim 17, wherein the customizable flange comprises a receiving member, and wherein receiving member is configured to couple to a first flange member and to a second flange member.

20. The assembly of claim 17, wherein the customizable flange comprises a first material and a second material, and wherein the first material comprises less mechanical strength than the second material, and wherein the first material is configured to be cut to customize the second length, the second width, or the combination thereof.
